(19) United States
(12) Patent Application Publication
(10) Pub. No.: US 2009/0221910 A1
(43) Pub. Date: Sep. 3, 2009

(54) LOCATION AND STABILIZATION DEVICE

(76) Inventors: George Lee Bulbrook, Norfolk (GB); David Paul Dearnaley, Surrey (GB)

Correspondence Address: RENNER OTTO BOISSELLE & SKLAR, LLP, 1621 EUCLID AVENUE, NINETEENTH FLOOR, CLEVELAND, OH, 44115, US

(21) Appl. No.: 12/089,981

(22) PCT Filed: Oct. 9, 2006
(86) PCT No.: PCT/GB2006/003739

§ 371 Date: Aug. 11, 2008

(30) Foreign Application Priority Data
Oct. 11, 2005 (GB) 0520596.8

(51) Int. Cl. A61B 5/05

(52) U.S. Cl. 600/426

(57) ABSTRACT

A prostate gland location and stabilization device comprising an elongate cylinder, sized to fit in the anal canal of a user, and having either a narrowing, or a bend (in the range from 20 to 60 degrees) located at or towards the axial mid-region of the cylinder. The cylinder has a heterogeneous internal structure arranged to define landmarks detectable by X-ray or magnetic resonance imaging. Additional features include an essentially flat handle, an internal gas channel and contrast markers. In some variants of the device, the heterogeneous structure comprises air-filled pockets. The invention also provides a method of locating the prostate gland of a patient using the device.

### FIELD OF THE INVENTION

The invention relates to devices to assist a clinician in locating the position of an internal organ of a patient, especially the prostate gland, by use of non-invasive imaging.

### BACKGROUND

Prostate cancer is the most common cancer of men in the developed world. Following diagnosis, several treatment options are available; one of these is radiotherapy. During treatment, the tumour must be exposed to radiation to treat the cancerous cells, and it is therefore important that an attending clinician can locate the position of the prostate. Accurate location of the prostate is important to reduce the risk of damage to adjacent tissues, such as the walls of the anus, rectum, and colon.

One technique that has been used to assist with this process is the insertion of a number of gold “seeds” into the prostate. The seeds—each about the size of a grain of rice—are put in place, usually with the help of ultrasound imaging, using long hollow needles through which the seeds are injected. The gold seeds are opaque to X-rays, and so may be used to “triangulate” the position of the prostate. The prostate is not usually visible on X-ray images, but computer tomography may be used after implantation of the seeds, to image both the seeds and the prostate, to determine the position of the seeds relative to the prostate gland. Having established the relationship between the seeds and the gland, conventional X-ray imaging may then be used to locate the seeds and therefore infer the position of the prostate. The requirement to implant the gold seeds is clearly invasive, often leading to an initial inflammation of the prostate, and demands skilled resources to locate the seeds before therapy can begin.

Another problem that can occur is instability of the position of the prostate gland during treatment. The prostate lies adjacent the wall of the rectum, and any gas build-up in the rectum or colon during treatment can displace the position of the prostate.

As a result of these difficulties, it is difficult to accurately judge the position of the prostate gland, and common practice is to irradiate a slightly larger target area to ensure that the tumour receives an adequate dose of radiation. This can increase the risk of inadvertent damage to the adjacent rectal wall.

The present invention attempts a solution to some of these problems.

### SUMMARY OF THE INVENTION

Accordingly, the invention provides a location and stabilization device comprising: an elongate cylinder, sized to fit in the anal canal and rectum of a user; and the interior of said cylinder having a heterogeneous internal structure arranged to define landmarks detectable by X-ray or magnetic resonance imaging. The device is particularly suitable for stabilizing and locating the position of the prostate gland, although, with small changes in geometry, would also be suitable for locating and stabilizing other organs or structures lying close to the anal canal such as the bladder or other pelvic organs. For clarity, the invention will be described in relation to its use with respect to the prostate. The cylinder may have various cross-sectional profiles, not necessarily circular, nor even symmetric, but for the comfort of the patient, and ease of insertion, a circular or oval cross-section is to be preferred.

Preferably, the cylinder has a bend in the range from 20 to 60 degrees, said bend being located at or towards the axial mid-region of the cylinder.

The bend is located in the cylinder to match the profile of the internal anatomy of the anal canal and rectum when the device is in position. When in position, the bend aligns such that it is adjacent to the position of the prostate gland, the bend effectively “pointing to” the prostate. For adult human use, the bend is ideally located so it lies approximately between 30 and 50 mm internal of the anal sphincter when the device is in position. The diameter of the cylinder is preferably of such as dimension as to lie flush against the internal wall of the rectum and anus, and preferably to slightly distend it. In this way, the wall of the colon is slightly stretched and consequently moved partially away from the prostate—i.e. from the site of radiotherapy treatment. This has the effect of reducing inadvertent damage to the rectum and anal wall. An appropriate diameter (for adult human use) would be in the range of 10 to 25 mm, with a diameter of approximately 15 mm being particularly appropriate. The heterogeneous internal structure may comprise differing materials that show different opacities to X-rays or that show contrast in magnetic resonance imaging, or other imaging system to be used. For example, metal inserts within a plastics body may be used to provide contrast for X-ray imaging, and an aqueous gel within a plastics body would provide contrast for MRI. A particularly preferred aspect where the heterogeneity is provided by air-filled voids is described below.

Preferably, the angle of the bend is approximately 40 degrees. This corresponds to an “average” anatomy, and makes one design of the device usable with a range of patient anatomies.

Alternatively, the cylinder is narrowed at or towards the axial mid-region of the cylinder. Such a feature (as with the bend, described above) also serves to locate the device adjacent an internal structure of interest—such as the prostate gland.

Also, in any aspect of the invention it is preferable that the cylinder is tapered. Such a tapered construction allows the device to be inserted more easily into the anus (narrow end first) without trauma to the sphincter.

Also, in any aspect of the invention it is preferable that the device further comprises a handle at one end of the cylinder; the handle being essentially flat and having a mid-plane lying in or about the longitudinal mid-plane of the bent cylinder. Whilst the provision of a handle at one end makes the device easier to remove, particular advantages spring from an essentially flat construction: Once inserted into the anus, the handle ties between the buttocks of a patient, and is effectively gripped there. This provides a resistance to any rotational movement of the device and acts to ensure that the device is located in the same position each time it is used.

Also, in any aspect of the invention it is preferable that the device has an internal channel providing gaseous communication from one end to the other. It is known that a build-up of gas is Likely to occur in the colon and rectum and, if this occurs during the treatment cycle, it can distend the colon and rectal wall, leading to a displacement of the prostate. With the device in place, build-up of flatus could also displace the device itself. The provision of a gaseous communication channel from one end to the other allows the gas to escape to atmosphere.

Also, in any aspect of the invention it is preferable that the device has a constriction between the cylinder and the handle. Provision of such a constriction renders the device “self-locating”: as the anal sphincter contracts after the device is inserted, it grips the device at the location of the constriction thus anchoring it at the correct—and repeatable—depth. It is preferable that the sides of the constriction are angled at between approximately 45 and 60 degrees for secure location, and ease of removal of the device. The corners of the constriction have smooth transitions, again for ease of removal of the device and comfort of the patient.

Also, in any aspect of the invention it is preferable that the landmarks include a linear feature aligned to substantially bisect the angle defined by the bent cylinder. Provision of such features gives a clear indication to the attending clinician of the position of the prostate in relation to the device.

Also, in any aspect of the invention it is preferable that the heterogeneous internal structure comprises air-filled voids. Air-filled voids provide good contrast on X-Ray and MRI images, and the use of air as the second material in the internal structure facilitates manufacture and reduces overall cost of the device. In a particularly preferred embodiment, the device may be manufactured from plastics by injection moulding. High impact polystyrene is a particularly appropriate material as it has appropriate mechanical properties, and has been found to be mucous membrane acceptable.

More preferably, the device is of separable two-part construction, so allowing access to the internal voids. With this feature, contrast markers may be inserted into the voids. Where X-rays are to be used for imaging the device, contrast markers in the form of strips of metal may be inserted into the air-filled voids, to further facilitate imaging of the landmarks. Where other imaging techniques are to be used, alternative materials for contrast markers will be evident to the skilled addressee. In particularly advantageous embodiments, the two parts are connectable by hollow studs adapted to receive contrast markers. In a related advantageous development, the internal (air-filled) voids define pockets adapted to receive contrast markers.

The scope of the invention includes, therefore, such a device further comprising one or more contrast markers. It is particularly advantageous that at least one contrast marker is in the form of a ring. The benefit of a ring-shaped marker is that its image gives information on the orientation of the marker. For example, if a circular ring is used, when imaged “face-on” it wilt appear as a circle; if the device is tilted with respect to the image plane, the image of the marker wilt become progressively more oval in shape. Similarly, if the ring is viewed “end-on” the image wilt be of a line; if the device is tilted with respect to the image plane, the image wilt become progressively more oval.

Also included within the scope of the invention is a prostate gland location and stabilization device substantially as described herein, with reference to, and as illustrated by, any appropriate combination of the accompanying drawings.

Also included within the scope of the invention is a method of locating the prostate gland of a patient on multiple occasions comprising the steps of: inserting a prostate gland location and stabilization device as described above into the anus of a patient; using an imaging system to obtain an image of the device's landmarks and the prostate gland; determining and recording the spatial relationship between the landmarks and the prostate gland; and on a subsequent occasion: inserting a prostate gland location and stabilization device as described above into the anus of a patient; using an imaging system to obtain an image of the device's landmarks; and using said previously recorded spatial relationship to determine the location of the prostate gland.

### BRIEF DESCRIPTION OF THE DRAWINGS

The invention will be described with reference to the accompanying drawings, in which:

FIG. 1 is a cross-sectional view of a device for location and stabilisation of the prostate gland;

FIG. 2 is a schematic diagram of a device showing a linear Landmark;

FIG. 3 illustrates images of a ring-shaped contrast marker;

FIG. 4 illustrates corresponding two halves of a 2-part separable construction of a device;

FIG. 5 illustrates the detail of a connecting stud, adapted to receive a contrast marker;

FIG. 6 and FIG. 7 illustrate the positioning of contrast markers within the body of a device;

FIG. 8 illustrates an alternative design of handle for a device;

FIG. 9 is a schematic diagram illustrating the device in use;

FIG. 10 is an end view of the device, in use;

FIG. 11 illustrates a device having a narrowed cylinder.

### DESCRIPTION OF THE PREFERRED EMBODIMENTS

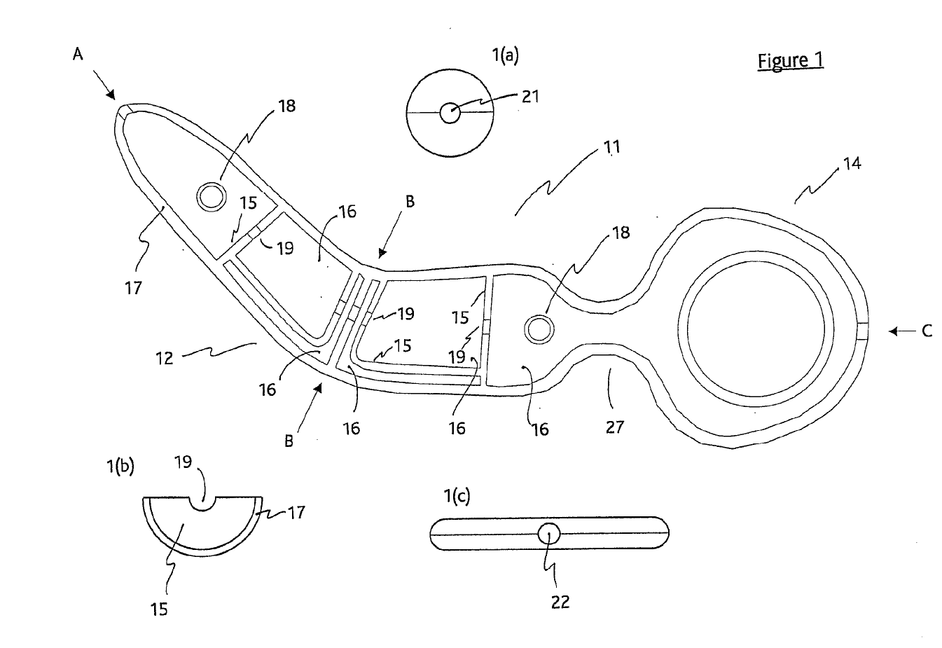

FIG. 1 illustrates a cross-section through a prostate gland location and stabilisation device, generally indicated by 11, according to the present invention. The device comprises an elongate cylinder 12 having a bend 13 located towards the axial mid-region of the cylinder 12. The device has a handle 14 at one end of the cylinder 12 enabling the device to be conveniently fitted in, and subsequently removed from, the anus of a patient. The heterogeneous internal structure is formed by the presence of a series of internal ribs 15 defining air-filled voids 16 between them, and between the ribs 15 and the outer shell 17 of the device 11.

The illustration shows one half of a 2-part construction, and two connecting studs 18 are also illustrated. The studs 18 are hollow to enable contrast markers to be located within the studs, if required. This feature is discussed in more detail below.

The device has an internal channel providing gaseous communication from one end to the other. In this embodiment, this is achieved by providing a series of cut-outs 19 in the ribs 15, so connecting air-fitted void 16 to form a channel from one end to the other. FIG. 1 (a) is an end view of device along the line of arrow A and shows a hole 21 formed in the outer shell 17 of the device 11 providing gaseous communication between the interior and exterior of the device 11. In particularly preferred embodiments, more than one hole is provided at this end on the device, preferably located at or adjacent the tip of the device; the provision of multiple holes ensures that the gas communication channel remains open, and not blocked by faecal material. (FIG. 1 (b) is a cross-section along the Line B-B of one half of the 2-part device and illustrating the form of the cut-out 19 in the rib 15. FIG. 1 (c) is an end view of the handle 14 along the line of arrow C. The view shows a second hole 22 formed in the outer skin 17 of the device, again providing gaseous communication between the interior and exterior of the device 11.

The device 11 has a constriction 27 located between the handle 14 and the cylinder 12. As discussed above, the constriction is so shaped and sized as to be gripped by the anal sphincter muscle of the patient when the device is in use. This provides a secure and, more importantly, reproducible means of locating the device in relation to the prostate gland.

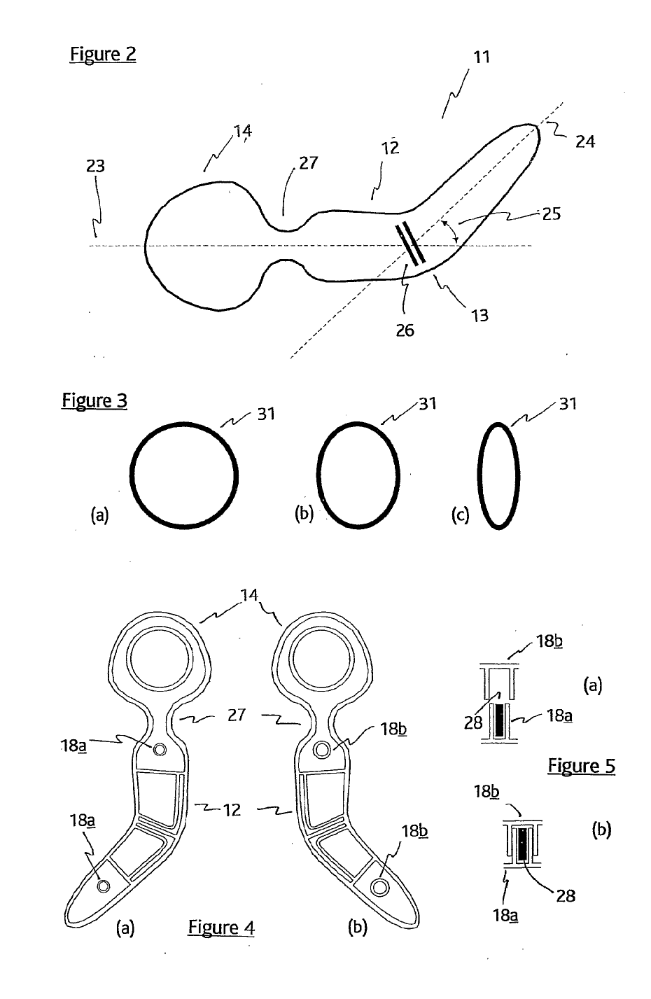

FIG. 2 is a schematic diagram of a device 11 having a tapered cylinder 12 with a bend 13 towards its axial mid-region, and a handle 14. The bend 13 in the device defines two axes 23 and 24 defining an angle of bend 25. Together, the two non-parallel axes 23 and 24 define a longitudinal mid-plane of the bent cylinder. Internal landmarks 26 in this device are of linear form, and are aligned to substantially bisect the obtuse angle defined by the bent cylinder.

FIG. 4 illustrates two corresponding halves of a device formed as a 2-part construction. Two pairs of interacting studs 18a and 18b are illustrated, to clip the two halves of the device together. FIG. 5 illustrates a cross-section through one pair of lugs 18 initially in the unconnected configuration, FIG. 5 (a), and subsequently when clipped together, FIG. 5 (b). It can be seen at the lugs 18 are hollow, enabling a contrast marker 28 to be located within the stud.

FIG. 6 illustrates the use of additional contrast markers 29 in the device 11. FIG. 6 (a) shows, in the plan view, a pair of contrast markers 29 formed from a strip of metal. FIG. 6 (b) illustrates the positioning of the contrast markers 26 in an air-filled void between ribs 15 and the outer shell 17 of the device. In this instance, it can be seen that the contrast markers 29 define an internal landmark, of substantially linear form, and bisecting the obtuse angle defined by the bent cylinder.

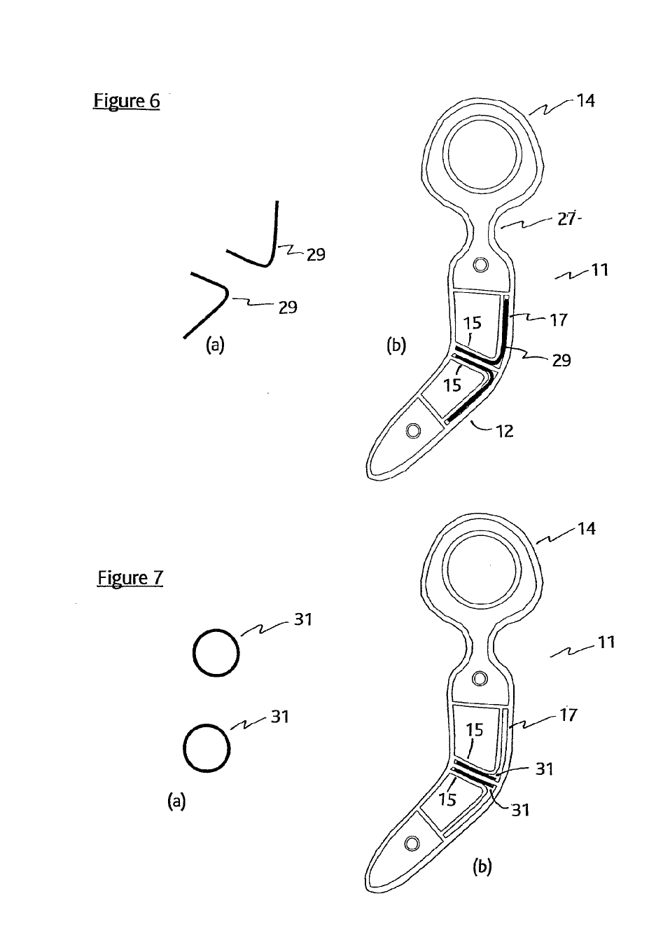

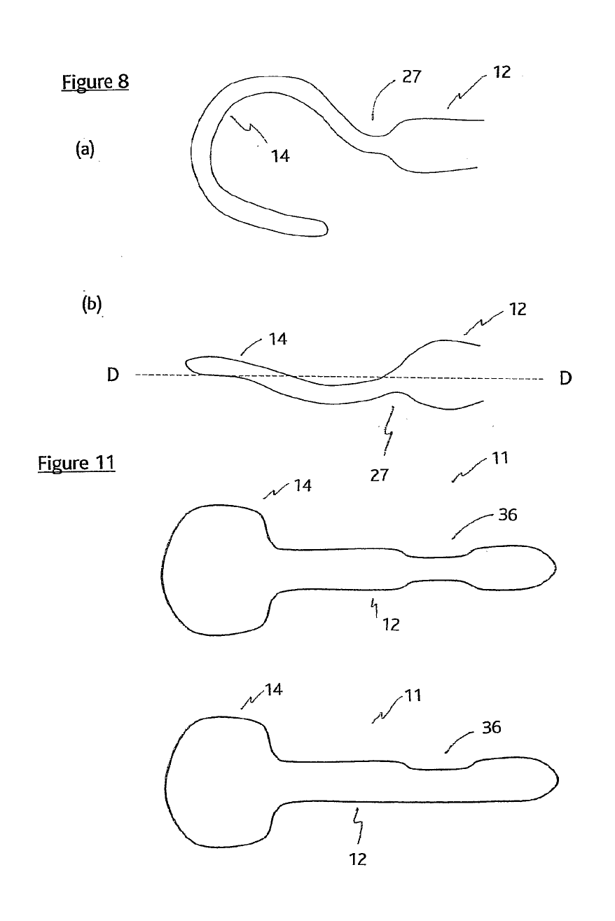

FIG. 7 illustrates the use of contrast markers 31 each in the form of a ring. Again, the markers 31 may be formed of a suitable material to give a high contrast on the imaging system, e.g. maybe formed of metal for x-ray imaging. In this instance, the ring-shaped contrast markers 31 are inserted into the interior of the device, and located in pockets defined by adjacent ribs 15. Again, they define a linear feature, bisecting the obtuse angle formed by the bent cylinder. The particular advantage of using ring-shaped contrast markers 31 is illustrated in FIG. 3. FIG. 3 (a) illustrates an image of the marker 31 when viewed “end on”, i.e. parallel to the image plane. FIGS. 3 (b) and 3 (c) illustrate the resultant image as the device is twisted with respect to an image plane. The changing shape allows a radiographer or radiologist to determine, therefore, whether the device is aligned as required.

FIG. 8 illustrates an alternative design of handle 14 connected to the cylinder 12 (shown in part). FIG. 8 (a) is a plan view and FIG. 8 (b) is a side view. It can be seen from FIG. 8 (b) that the handle 14 is essentially flat, and defines a mid-plane indicated by the dashed line D-D.

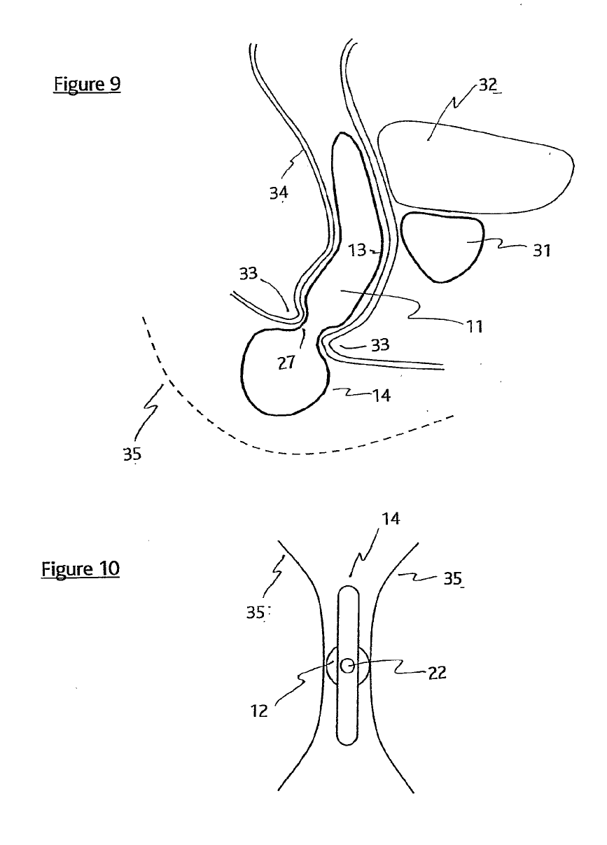

FIG. 9 is a schematic cross-section of a device 11 in situ in the anal canal of a patient. The diagram illustrates the position of the prostate gland 31, the bladder 32, the location of the anal sphincter 33, the wall of the colon 34 and the outline of the patient's buttocks 35. It can be seen that the device follows the contour of the wall of the colon 34, with the bend 13 of the cylinder lying adjacent the prostate gland 31. As discussed above, the diameter of the device is such that it sits against the colon wall 34, and preferably distends it slightly, to stretch the colon wall tissue away from the site of treatment in the prostate gland 31. The diagram illustrates how the constriction 27 in the device 11 is gripped by the anal sphincter 33 to positively locate the device in the desired position. The diagram also illustrates how the handle 14 of the device sits between the buttocks of a patient in use.

FIG. 10 is an end view of the handle 14 of the device in situ in the anus of a patient, illustrating how the essentially flat form of the handle 14 is gripped by the buttocks 35 of the patient. In use, the buttocks 35 would close to more closely grip the handle 14 than is illustrated in FIG. 10, for clarity. The resilient nature of the buttocks 35 mean that the handle 14 does not have to be absolutely flat, in the literal sense, but essentially flat, defining a mid-plane as illustrated in FIG. 8.

FIG. 11 illustrates the overall external shape of two variants of a location and stabilization device, generally indicated by 11, each comprising a cylinder 12 that is narrowed towards the axial mid-region of the cylinder. The narrowing 36 is located to lie adjacent an organ of interest. The internal landmark features are not illustrated, for clarity.

1. A location and stabilization device, to assist a clinician in locating the position of an internal organ of a patient, comprising:
an elongate cylinder, sized to fit in the anal canal of a user;
the interior of said cylinder having a heterogeneous internal structure arranged to define landmarks detectable by X-ray or magnetic resonance imaging;
a handle at one end of the cylinder; and
said cylinder has a bend located at or towards the axial mid-region of the cylinder; and wherein there is provided a constriction between said cylinder and said handle, thereby anchoring the device, in use, at a repeatable depth in the anal canal of a user.

2. A device according to claim 1 wherein the cylinder has a bend in the range from 20 to 60 degrees, said bend being located at or towards the axial mid-region of the cylinder.

3. A device according to claim 2 wherein the angle of the bend is approximately 40 degrees.

4. (canceled)

5. A device according to claim 1 wherein the cylinder is tapered.

6. A device according to claim 1 wherein said handle is essentially flat and having a mid-plane lying in or about the longitudinal mid-plane of the bent cylinder.

7. A device according to claim 1 wherein the device has an internal channel providing gaseous communication from one end to the other.

8. (canceled)

9. A device according to claim 1 wherein the landmarks include a linear feature aligned to substantially bisect the angle defined by the bent cylinder.

10. A device according to claim 1 wherein the heterogeneous internal structure comprises air-filled voids.

11. A device according to claim 10 wherein the device is of separable two-part construction, so allowing access to the internal voids.

12. A device according to claim 11 wherein the two parts are connectable by hollow studs adapted to receive contrast markers.

13. A device according to claim 11 wherein the internal voids define pockets adapted to receive contrast markers.

14. A device according to claim 12, further comprising one or more contrast markers.

15. A device according to claim 14 wherein at least one contrast marker is in the form of a ring.

16. A location and stabilization device substantially as described herein, with reference to and as illustrated by any appropriate combination of the accompanying drawings.

17. A method of locating the prostate gland of a patient on multiple occasions comprising the steps of:
inserting a device according to claim 1 into the anus of a patient; using an imaging system to obtain an image of the device's landmarks and the prostate gland;
determining and recording the spatial relationship between the landmarks and the prostate gland;
and on a subsequent occasions, inserting a device according to claim 1 into the anus of a patient;
using an imaging system to obtain an image of the device's landmarks; and
using said previously recorded spatial relationship to determine the location of the prostate gland.
